(19) United States
(12) Patent Application Publication
(10) Pub. No.: US 2025/0003912 A1
(43) Pub. Date: Jan. 2, 2025

(54) NANOGAP-BASED HYDROGEN SENSOR AND MANUFACTURING METHOD THEREOF

(71) Applicant: KOREA INSTITUTE OF SCIENCE AND TECHNOLOGY, Seoul (KR)

(72) Inventors: Minah SEO, Seoul (KR); Yong-Sang RYU, Seoul (KR); Jinwoo LEE, Seoul (KR); Geon LEE, Seoul (KR); Jongsu LEE, Seoul (KR)

(21) Appl. No.: 18/746,488

(22) Filed: Jun. 18, 2024

(30) Foreign Application Priority Data
Jun. 27, 2023 (KR) 10-2023-0082916

(51) Int. Cl. G01N 27/12; G01N 27/407

(52) U.S. Cl. CPC G01N 27/127; G01N 27/4075

(57) ABSTRACT

The object of the present invention is to provide a nanogap-based hydrogen sensor in which a nanogap is formed on a substrate and a trace amount of hydrogen gas leakage can be detected in a non-contact manner using the nanogap, and a method of manufacturing the same. In order to achieve the above object, the present invention is characterized by including: a metamaterial manufactured by forming a metal nanoslot pattern on a wafer; a catalyst layer deposited on the surface of the metamaterial to form a nanogap inside the metal nanoslot; and a protective layer formed on the catalyst layer to protect the catalyst layer.

### CROSS REFERENCE TO RELATED APPLICATION

The present application claims priority to Korean Patent Application No. 10-2023-0082916, filed on Jun. 27, 2023, the entire contents of which is incorporated herein for all purposes by this reference.

### BACKGROUND OF THE INVENTION

### Field of the Invention

The present invention relates to a nanogap-based hydrogen sensor and a method of manufacturing the same, and more specifically, to a nanogap-based hydrogen sensor in which a nanogap is formed on a substrate and a trace amount of hydrogen gas leakage can be detected in a non-contact manner using the nanogap, and a method of manufacturing the same.

### Description about National Research and Development Support

This study was supported by the technology development programs of Ministry of Science and ICT, Republic of Korea (Projects No. 1711196504 and No. 1711181827) under the Korea Institute of Science and Technology.

### Description of the Related Art

Recently, interest in hydrogen energy has increased, and anxiety about hydrogen gas, which is highly flammable and explosive, is also increasing.

Accordingly, the development of sensors that can quickly detect leaking hydrogen gas is also attracting a lot of attention.

Hydrogen sensors are made in an indirect method of measuring changes caused by the interaction between sensor material and hydrogen, and palladium (Pd) is widely used in hydrogen sensors because it is stable and shows low hydrogen separation activation energy.

When exposed to a hydrogen environment, palladium metal has the property of inducing a catalytic reaction and allowing hydrogen atoms to penetrate into the metal. The penetrance responds qualitatively to the concentration of surrounding hydrogen, and when the penetrance is high, the palladium metal has characteristics such as phase transition, volume expansion, and physical property changes.

FIG. 1 is a diagram illustrating a phase change of palladium due to hydrogen intrusion, in which palladium causes a phase change by hydrogen intrusion, and a hydrogen sensor detects hydrogen by measuring a change in physical properties according to this phase change.

Hydrogen detection methods using palladium-based hydrogen sensors include electrical and optical methods.

The electrical method is a technology based on a mechanism of volume expansion according to the phase change of palladium, and is a technology that measures a difference in electrical signals due to contact between palladium fine particles caused by the volume expansion (see FIG. 2). Because it is an electrical measurement method, it has a fast response time and sensitivity, but because it requires electrodes, electrode patterning is essential, contact is inevitable, and there is a safety issue that may cause an explosion due to leakage current.

The optical method is a technology based on a mechanism of volume expansion and optical constant value change according to the phase change of palladium, and is a technology that measures a signal change caused by a structural change and an optical constant change according to the phase change (see FIG. 3). The optical method has high sensitivity because it measures using plasmonics, but has a safety problem of explosion risk because light with a relatively short wavelength (high energy) is used.

### DOCUMENTS OF RELATED ART

### Patent Documents

(Patent Document 1) Korean Registered Patent No. 10-1497339 (registered on Mar. 5, 2015).

### SUMMARY OF THE INVENTION

### Technical Problem

The present invention has been devised to solve the problems of the prior art as described above, and aims to provide a nanogap-based hydrogen sensor in which a nanogap is formed on a substrate and a trace amount of hydrogen gas leakage can be detected in a non-contact manner using the nanogap, and a method of manufacturing the same.

### Technical Solution

A nanogap-based hydrogen sensor according to the present invention for achieving the above-described object include: a metamaterial manufactured by forming a metal nanoslot pattern on a wafer; a catalyst layer deposited on the surface of the metamaterial to form a nanogap inside the metal nanoslot; and a protective layer formed on the catalyst layer to protect the catalyst layer.

Additionally, in the nanogap-based hydrogen sensor according to the present invention, when the catalyst layer is formed by depositing a catalyst material on the surface of the metamaterial, the nanogap may be formed between the metal wall surface and the catalyst layer inside the metal nanoslot due to the step difference of the metal nanoslot.

In addition, in the nanogap-based hydrogen sensor according to the present invention, the nanogap may have a width of 10 to 25 nm.

In addition, in the nanogap-based hydrogen sensor according to the present invention, the catalyst layer may cause a distance change from the wall surface of the metal forming the metal nanoslot as the volume thereof expands due to a reaction with hydrogen, thereby changing a gap size of the nanogap.

In addition, in the nanogap-based hydrogen sensor according to the present invention, the metal nanoslot pattern formed on the wafer may be formed through a photolithography process.

Further, in the nanogap-based hydrogen sensor according to the present invention, the catalyst layer may be formed by depositing a catalyst material on the surface of the metamaterial through a thermal evaporator.

In addition, a method of manufacturing a nanogap-based hydrogen sensor according to the present invention for achieving the above-described object, the method including the steps of: manufacturing a metamaterial by forming a metal nanoslot pattern on a wafer; depositing a catalyst material on the surface of the metamaterial to form a catalyst layer forming a nanogap inside the metal nanoslot; and forming a protective layer protecting the catalyst layer on the catalyst layer.

Additionally, in the method of manufacturing a nanogap-based hydrogen sensor according to the present invention, when the catalyst layer is formed by depositing a catalyst material on the surface of the metamaterial in the step of forming the catalyst layer, the nanogap may be formed between the metal wall surface and the catalyst layer inside the metal nanoslot due to the step difference of the metal nanoslot.

In addition, in the method of manufacturing a nanogap-based hydrogen sensor according to the present invention, the step of manufacturing the metamaterial may be a step of manufacturing the metamaterial by forming the metal nanoslot pattern on the wafer through a photolithography process.

Additionally, in the method of manufacturing a nanogap-based hydrogen sensor according to the present invention, the step of forming the catalyst layer may be a step of forming a catalyst layer forming a nanogap inside the metal nanoslot by depositing a catalyst material on the surface of the metamaterial through a thermal evaporator.

Specific details of other embodiments are included in the “DETAILED DESCRIPTION OF THE INVENTION” and the attached “DRAWINGS”.

The advantages and/or features of the present invention, and methods for achieving them will become clear with reference to various embodiments described in detail below along with the accompanying drawings.

However, the present invention is not limited to the configuration of each embodiment described below, but may also be implemented in various different forms. Each of the embodiments disclosed in this specification is provided only to make the disclosure of the present invention complete and to fully inform those skilled in the art of the scope of the invention. It should be noted that the present invention is only defined by the scope of each of the claims.

### Advantageous Effects

According to the present invention, it is possible to detect a trace amount of hydrogen gas leakage through the nanogap formed between the catalyst layer and the metal wall surface.

In addition, since a beam in the terahertz band is irradiated to a hydrogen sensor with a nanogap formed and a signal transmitted through the hydrogen sensor is measured to detect hydrogen leakage, electrode patterning is not required and a high detection speed of less than tens of seconds is achieved.

In addition, since a micrometer-scale metal nanoslot pattern is formed on the wafer through a photolithography process, and a catalyst material is deposited on the surface of the metamaterial through a thermal evaporator, it is possible to minimize differences between devices and mass-produce devices with uniform performance.

Additionally, it is possible to inversely estimate the concentration of hydrogen by measuring the shift of resonance frequency with low noise.

In addition, one nanogap-based hydrogen sensor can be used to detect not only hydrogen but also oxygen.

### BRIEF DESCRIPTION OF THE DRAWINGS

FIG. 1 is a diagram illustrating a phase change of palladium due to hydrogen intrusion.

FIGS. 2 and 3 are diagrams illustrating a hydrogen detection method using a palladium-based hydrogen sensor according to the prior art.

FIGS. 4 and 5 are diagrams schematically showing the structure of a nanogap-based hydrogen sensor according to an embodiment of the present invention.

FIG. 6 is a diagram exemplarily illustrating a metamaterial applied to the present invention.

FIG. 7 is a diagram for explaining a method of manufacturing a nanogap-based hydrogen sensor according to an embodiment of the present invention.

FIG. 8 is a diagram exemplarily showing that the volume of a catalyst layer applied to the present invention expands as it is exposed to hydrogen.

FIGS. 9 and 10 are diagrams schematically showing the configuration of a hydrogen detection system using a nanogap-based hydrogen sensor according to an embodiment of the present invention.

FIGS. 11, 12, and 13 are diagrams exemplarily showing field enhancement formed by a nanogap according to the present invention.

FIG. 14 is a diagram exemplarily illustrating a change in a signal of a terahertz beam received through a first optical path (A) according to hydrogen concentration.

FIG. 15 is a diagram exemplarily showing changes in a resonance frequency and a transmittance value when a catalyst layer is exposed to hydrogen.

FIG. 16 is a diagram for explaining oxygen detection using a nanogap-based hydrogen sensor according to the present invention.

FIGS. 17A, 17B, 17C, and 17D are diagrams exemplarily illustrating the dynamics of water generation.

FIGS. 18A, 18B, 18C, and 18D and FIGS. 19A, 19B, 19C, 19D, 19E, and 19F are diagrams exemplarily illustrating dynamics analysis using a frequency change and a transmittance change as variables according to an oxygen concentration change.

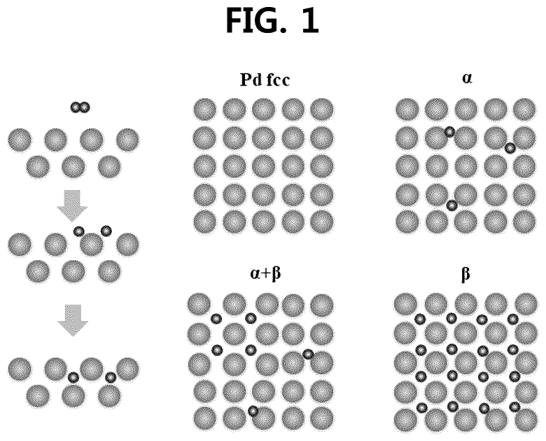

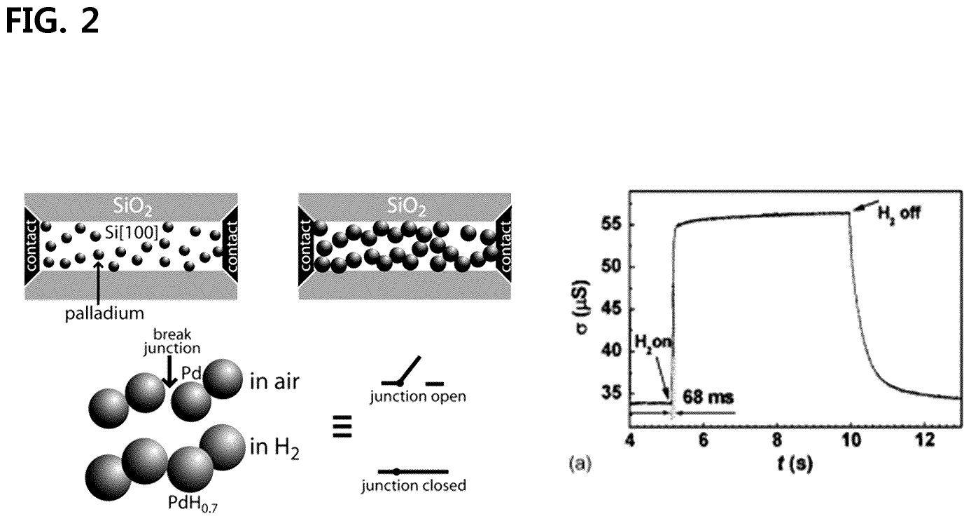

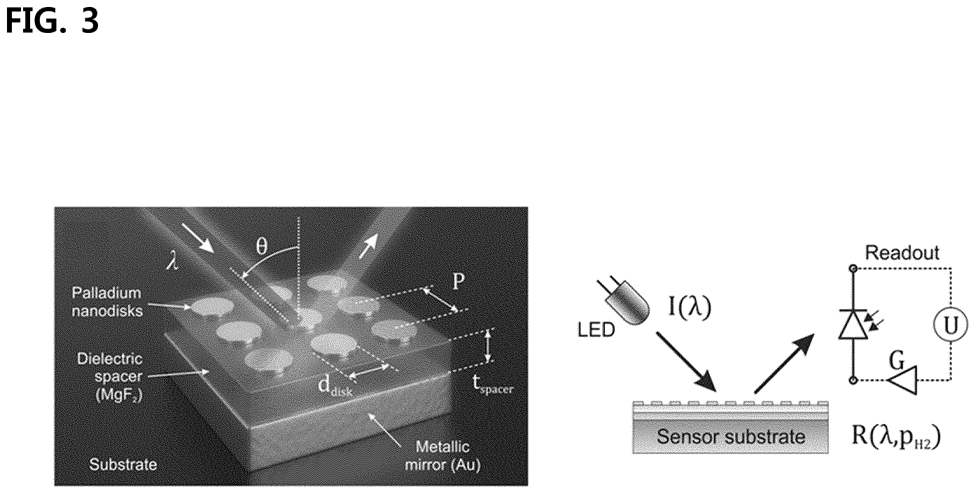

### DETAILED DESCRIPTION OF THE INVENTION

Before describing the present invention in detail, it should be noted that the terms or words used in the present specification should not be construed as unconditionally limited to their ordinary or dictionary meanings, and that the present inventors may appropriately define and use the concepts of various terms in order to explain their invention in the best way, and furthermore, these terms or words should be interpreted as meanings and concepts consistent with the technical spirit of the present invention.

In other words, it should be noted that the terms used in the present specification are only used to describe preferred embodiments of the present invention, are not intended to specifically limit the contents of the present invention, and are defined in consideration of various possibilities of the present invention.

In addition, it should be noted that in this specification, singular expressions may include plural expressions unless clearly indicated in a different meaning in the context, and similarly, even if the expressions are expressed in plural, they may include singular meanings.

Throughout this specification, when a component is described as “including” another component, it may mean that any other component may be further included, rather than excluded, unless specifically stated to the contrary.

Furthermore, it should be noted that when a component is described as being “installed inside or connected to” another component, this component may be installed in direct connection or contact with the other component and may be installed at a certain distance, and that when installed at a certain distance, there may be a third component or means for fixing or connecting the component to another component, and the description of this third component or means may be omitted.

On the other hand, when a component is described as being “directly connected” or “directly connected” to another component, it should be understood that no third component or means is present.

Likewise, other expressions describing the relationship between each component, such as “between˜” and “just between˜”, or “neighboring to˜” and “directly neighboring to˜”, should be interpreted as having the same meanings.

In addition, it should be noted that terms such as “one”, “another”, “one side”, “other side”, “first”, and “second” in this specification, if used, are intended to allow one component to be clearly distinguished from another component, and the meaning of that component is not limited by these terms.

In addition, in this specification, terms related to position such as “top”, “bottom”, “left”, “right”, etc., if used, should be understood as indicating relative positions in the drawing for the concerned component, and should not be understood as referring to absolute locations unless the absolute location is specified.

Moreover, it should be noted that terms such as “˜part”, “˜unit”, “module”, “device”, etc. in the specification of the present invention, when used, refer to a unit capable of processing one or more functions or operations, which can be implemented by hardware or software, or a combination of hardware and software.

In addition, in this specification, when indicating reference numerals for each component in each drawing, the same reference numeral is assigned to the same component even if the component is shown in different drawings. That is, the same reference numerals indicate the same components throughout the specification.

In the drawings attached to this specification, the size, position, coupling relationship, etc. of each component constituting the present invention may be partially exaggerated, reduced, or omitted in order to sufficiently clearly convey the spirit of the present invention or for convenience of explanation, and thus, the proportionality or scale may not be strict.

In addition, hereinafter, when describing the present invention, detailed descriptions of configurations that are judged to unnecessarily obscure the gist of the present invention, for example, known technologies including prior art, may be omitted.

Hereinafter, a nanogap-based hydrogen sensor and its manufacturing method according to a preferred embodiment of the present invention will be described in detail with reference to the attached drawings.

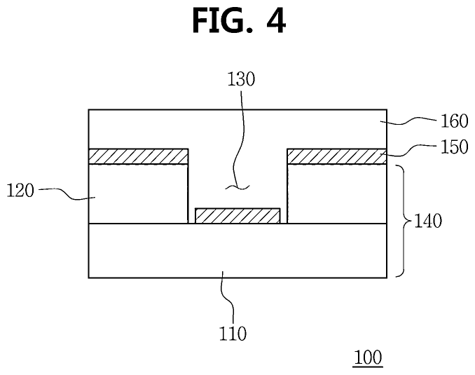

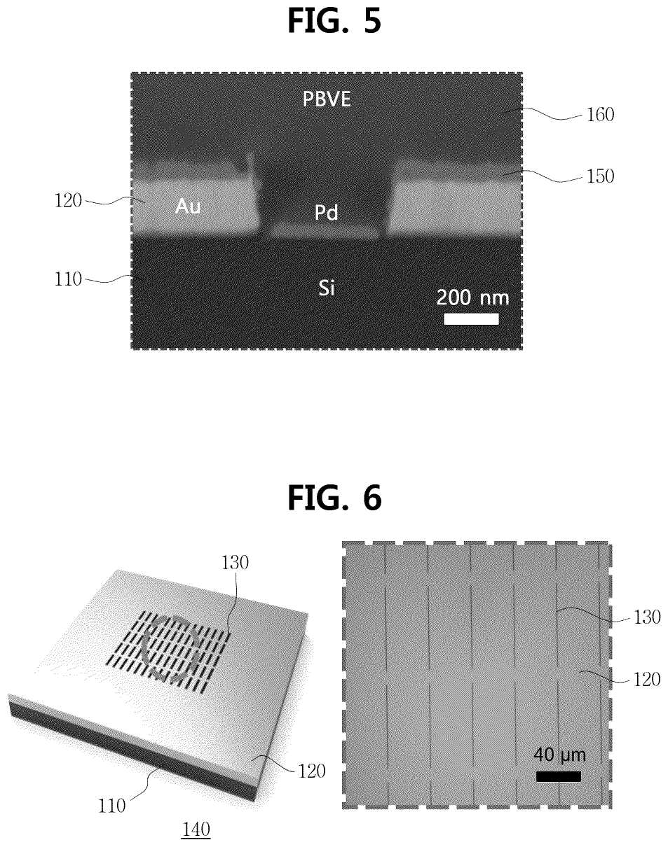

FIGS. 4 and 5 are diagrams schematically showing the structure of a nanogap-based hydrogen sensor according to an embodiment of the present invention.

As shown in FIGS. 4 and 5, the nanogap-based hydrogen sensor 100 according to an embodiment of the present invention may include a metamaterial 140, a catalyst layer 150, and a protective layer 160.

Here, the metamaterial 140 may be manufactured by forming a pattern of micrometer (μm) scale metal nanoslot 130 on a wafer 110, as shown in FIG. 6.

The pattern of metal nanoslot 130 formed on the wafer 110 may be formed through a photolithography process, but is not limited thereto.

When the pattern of micrometer-scale metal nanoslot 130 is formed on the wafer 110 through a photolithography process, a large amount of patterns of metal nanoslot 130 can be produced on a large wafer-scale area.

By adjusting the slot length and width of the pattern of metal nanoslot 130 formed on the wafer 110 through the photolithography process, the degree of field distribution and field enhancement, and the target resonance frequency can be adjusted.

In one embodiment of the present invention, gold (Au) may be used as the metal forming the metal nanoslot 130, but is not limited thereto.

Meanwhile, the catalyst layer 150 may be deposited on the surface of the metamaterial 140 to form a nanogap 155 inside the metal nanoslot 130.

Specifically, when a catalyst material is deposited on the surface of the metamaterial 140, the catalyst layer 150 is formed not only on the surface of the metal 120 but also inside the metal nanoslot 130. In this case, a nanogap 155 is formed between the catalyst layer 150 formed inside the metal nanoslot 130 and the wall surface of the metal 120 due to structural reasons caused by the step difference of the metal nanoslot 130.

As such, the nanogap 155 formed inside the metal nanoslot 130 may have a width of approximately 10 to 25 nm.

As the catalyst layer 150 expands in volume due to reaction with hydrogen, the distance from the wall surface of the metal 120 forming the metal nanoslot 130 changes, thereby changing the gap size of the nanogap 155.

The catalyst layer 150 may be formed by depositing a catalyst material on the surface of the metamaterial 140 through a thermal evaporator, but is not limited thereto.

In one embodiment of the present invention, the catalyst material deposited on the surface of the metamaterial 140 may be palladium (Pd) that expands in volume by reaction with hydrogen, but is not limited thereto.

When the catalyst layer 150 is implemented with palladium (Pd), the penetrance of hydrogen atoms into the palladium (Pd) catalyst layer 150 has an inherent phase transition characteristics of palladium metal, and is known to exhibit an alpha-phase at an ambient hydrogen concentration within approximately 1.25% and a beta-phase at a hydrogen concentration of approximately 2%, but the degree of reactivity may vary slightly depending on the volume and size of palladium metal.

Before depositing a catalyst material on the surface of the metamaterial 140 to form the catalyst layer 150 as described above, titanium (Ti) may be deposited on the surface of the metamaterial 140 through a thermal evaporator.

When titanium (Ti) is deposited, the nanogap-based hydrogen sensor 100 can continuously and repeatedly sense hydrogen.

Specifically, the titanium (Ti) improves high adhesion performance between the surface of the wafer 110 or metamaterial 140 at the bottom and the catalyst layer 150 applied on the top.

When exposed to hydrogen gas, the catalyst layer 150 expands in volume due to a reaction with the hydrogen gas, causing peeling from the surface of the lower wafer 110 or metamaterial 140. If peeling occurs repeatedly each time hydrogen is sensed, its performance as a hydrogen sensor may be seriously impaired. To prevent this, titanium (Ti) is placed between the surface of the lower wafer 110 or metamaterial 140 and the catalyst layer 150 applied thereon, thereby allowing the nanogap-based hydrogen sensor 100 to continuously and repeatedly sense hydrogen.

Meanwhile, the protective layer 160 may be formed on the catalyst layer 150 to protect the catalyst layer 150.

Specifically, the protective layer 160 may be formed on the catalyst layer 150 to allow hydrogen gas to selectively transmit and at the same time protect the catalyst layer 150 from the external environment.

The protective layer 160 may be formed by coating a polymer using a spin coating method, but is not limited thereto.

As described above, the protective layer 160 formed by spin coating a polymer can selectively transmit hydrogen gas and control the degree of evaporation of products caused by the catalytic reaction of the catalyst layer 150.

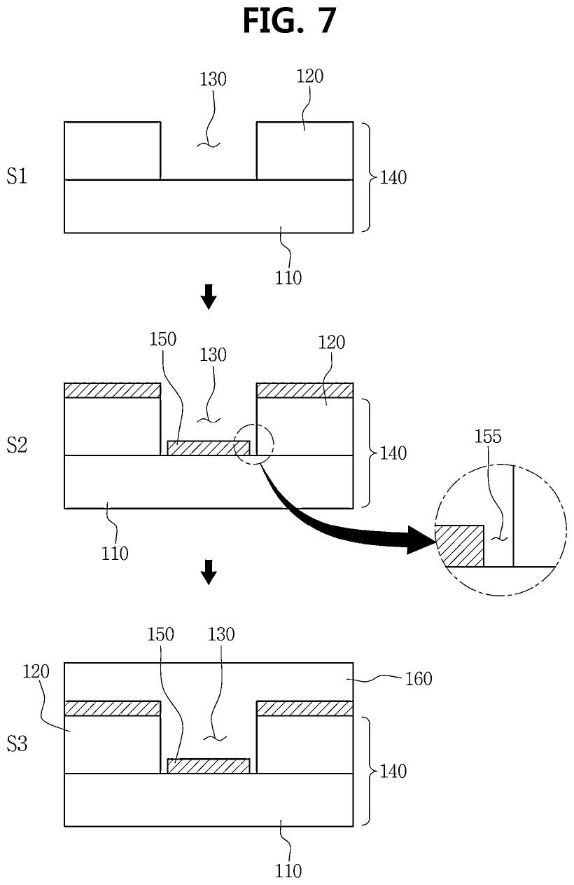

FIG. 7 is a diagram for explaining a method of manufacturing a nanogap-based hydrogen sensor according to an embodiment of the present invention.

First, a metamaterial 140 as shown in FIG. 6 can be manufactured by forming a pattern of micrometer-scale metal nanoslot 130 on a wafer 110 (S1).

In step S1 described above, the metamaterial 140 may be manufactured by forming a pattern of metal nanoslot 130 on the wafer 110 through a photolithography process, but is not limited thereto.

The slot length and width of the pattern of metal nanoslot 130 formed in step S1 above can be adjusted according to the degree of the target field distribution and field enhancement and the target resonance frequency.

Thereafter, a catalyst layer 150 forming a nanogap 155 inside the metal nanoslot 130 can be formed by depositing a catalyst material on the surface of the metamaterial 140 manufactured through step S1 above (S2).

Specifically, when the catalyst material is deposited on the surface of the metamaterial 140 in step S2 above, the catalyst layer 150 is formed not only on the surface of the metal 120 but also inside the metal nanoslot 130. In this case, the nanogap 155 is formed between the catalyst layer 150 formed inside the metal nanoslot 130 and the wall surface of the metal 120 due to structural reasons caused by the step difference of the metal nanoslot 130.

As such, the nanogap 155 formed inside the metal nanoslot 130 may have a width of approximately 10 to 25 nm.

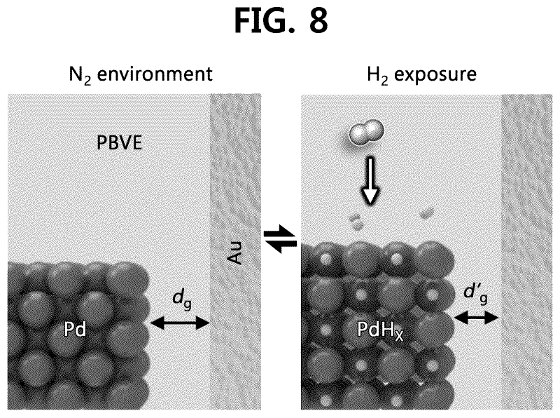

As the catalyst layer 150 formed through step S2 above undergoes a phase transition through reaction with hydrogen and expands in volume at an atomic level, the distance from the wall surface of the metal 120 forming the metal nanoslot 130 changes, thereby changing the gap size of the nanogap 155 at an atomic level, as shown in FIG. 8.

As described above, when the gap size of the nanogap 155 changes, the capacitance changes between the catalyst layer 150 and the wall surface of the metal 120, and hydrogen is detected through this change in capacitance. This will be described in more detail below.

In step S2 above, the catalyst layer 150 may be formed by depositing the catalyst material on the surface of the metamaterial 140 through a thermal evaporator, but is not limited thereto.

In one embodiment of the present invention, the catalyst material deposited on the surface of the metamaterial 140 may be palladium (Pd) that expands in volume by reaction with hydrogen, but is not limited thereto.

Before depositing the catalyst material on the surface of the metamaterial 140 to form the catalyst layer 150 in step S2 described above, titanium (Ti) may be deposited on the surface of the metamaterial 140 through a thermal evaporator. When the titanium (Ti) is deposited, continuous and repetitive sensing becomes possible.

Afterwards, a protective layer 160 protecting the catalyst layer 150 may be formed on the catalyst layer 150 (S3).

In step S3 above, the protective layer 160 may be formed by coating a polymer using a spin coating method, but is not limited thereto.

The protective layer 160 formed through step S3 above can allow hydrogen gas to selectively transmit, control the degree of evaporation of the product by the catalytic reaction of the catalyst layer 150, and at the same time protect the catalyst layer 150 from the external environment.

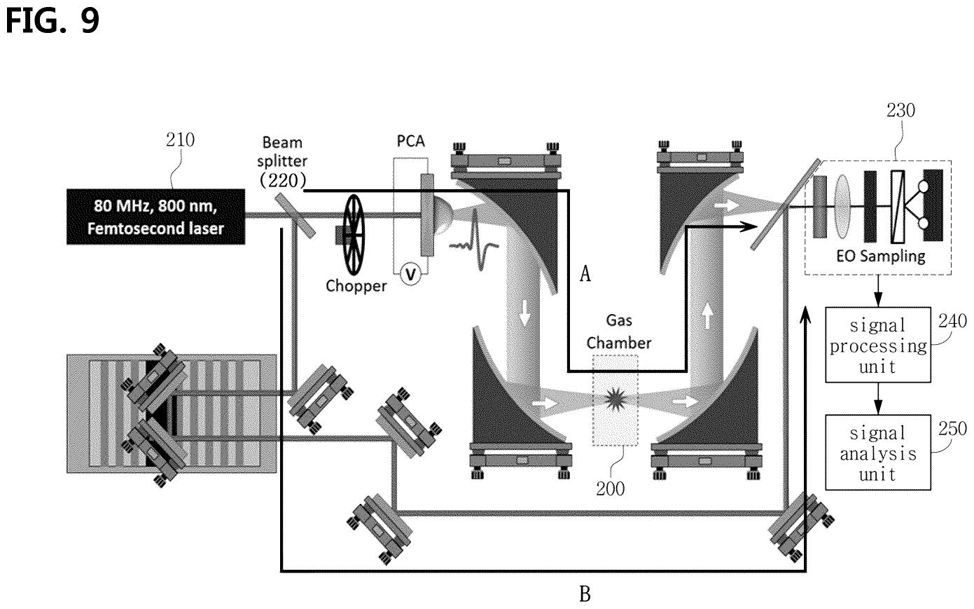

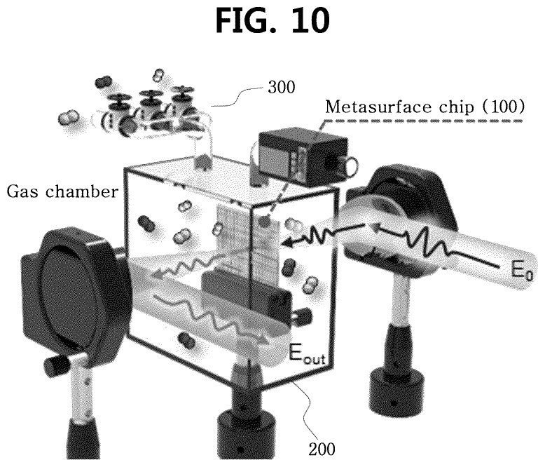

FIGS. 9 and 10 are diagrams schematically showing the configuration of a hydrogen detection system using a nanogap-based hydrogen sensor according to an embodiment of the present invention.

In an embodiment of the present invention, tera hertz (THz) time-based spectroscopy technology can be used to measure the distance change of the nanogap that changes as the hydrogen concentration increases. That is, hydrogen leakage can be detected by irradiating a beam in the terahertz (THz) band to the nanogap-based hydrogen sensor 100 and measuring the signal transmitted through the nanogap-based hydrogen sensor 100.

In an embodiment of the present invention, in order to measure the hydrogen detection ability of the nanogap-based hydrogen sensor 100 according to the present invention, the nanogap-based hydrogen sensor 100 may be mounted in a specially manufactured gas chamber 200, and gas may be quantitatively injected into the gas chamber 200 through the mass flow controller (MFC) 300, as shown in FIG. 10.

In addition, a femto second pulse laser with a wavelength of approximately 800 nm may be used to oscillate a terahertz (THz) pulse, and the oscillated terahertz beam may be irradiated to the nanogap-based hydrogen sensor 100 located within the gas chamber 200.

Specifically, a light source unit 210 may oscillate and output the terahertz (THz) pulses using the femto second pulse laser with a wavelength of 800 nm. Some of the terahertz beams output from the light source unit 210 may be transmitted to a first light path (A) by a beam splitter 220 and irradiated to the nanogap-based hydrogen sensor 100 located in the gas chamber 200, and the remainder of the beams may be reflected to a second optical path (B) by the beam splitter 220.

The terahertz beam transmitted through the beam splitter 220 and incident on the first optical path (A) may be irradiated to the nanogap-based hydrogen sensor 100, and the terahertz beam irradiated to the hydrogen sensor 100 may be transmitted through the hydrogen sensor 100 and incident on a beam receiving unit 230.

A plurality of reflectors (mirrors) are located on the first optical path (A), so that the terahertz beam transmitted through the beam splitter 220 may be irradiated to the hydrogen sensor 100, and the terahertz beam transmitted through the hydrogen sensor 100 may be incident on the beam receiving unit 230.

Meanwhile, the terahertz beam reflected from the beam splitter 220 and incident on the second optical path (B) may be incident on the beam receiving unit 230 through the second optical path (B) as it is.

A plurality of reflectors (mirrors) are located on the second optical path (B), so that the terahertz beam reflected from the beam splitter 220 may be incident on the beam receiving unit 230 as it is.

As described above, the beam receiving unit 230 may receive the terahertz beam transmitted through the hydrogen sensor 100 via the first optical path (A), and receive the terahertz beam output from the light source unit 210 through the second optical path (B) as it is.

Meanwhile, when the MFC 300 is controlled to inject hydrogen gas into the gas chamber 200 in which the nanogap-based hydrogen sensor 100 is mounted, the catalyst layer 150 formed in the nanogap-based hydrogen sensor 100 expands in volume by a reaction with hydrogen, resulting in a change in the distance between the wall surface of the metal 120 forming the metal nanoslot 130 and the catalyst layer 150.

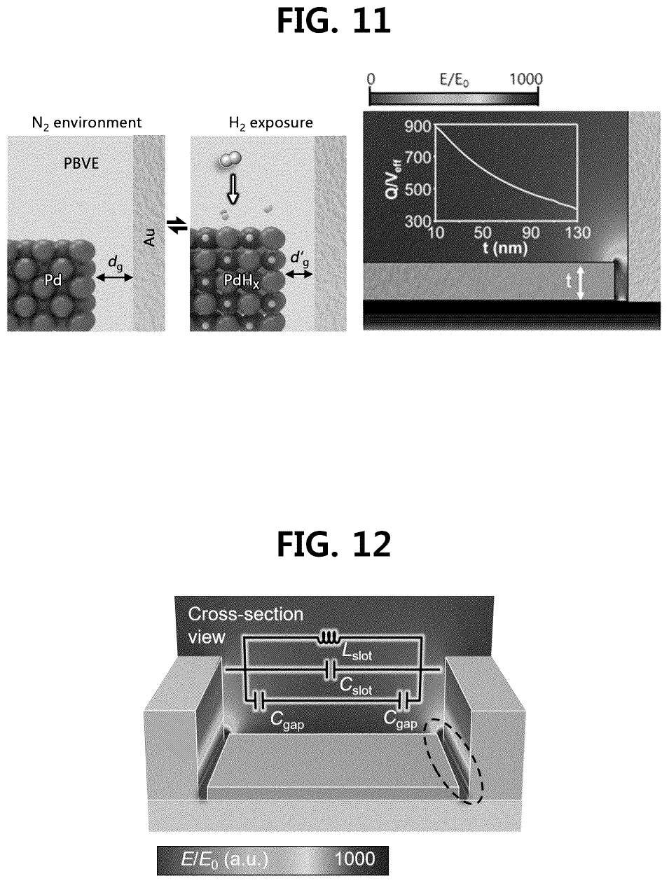

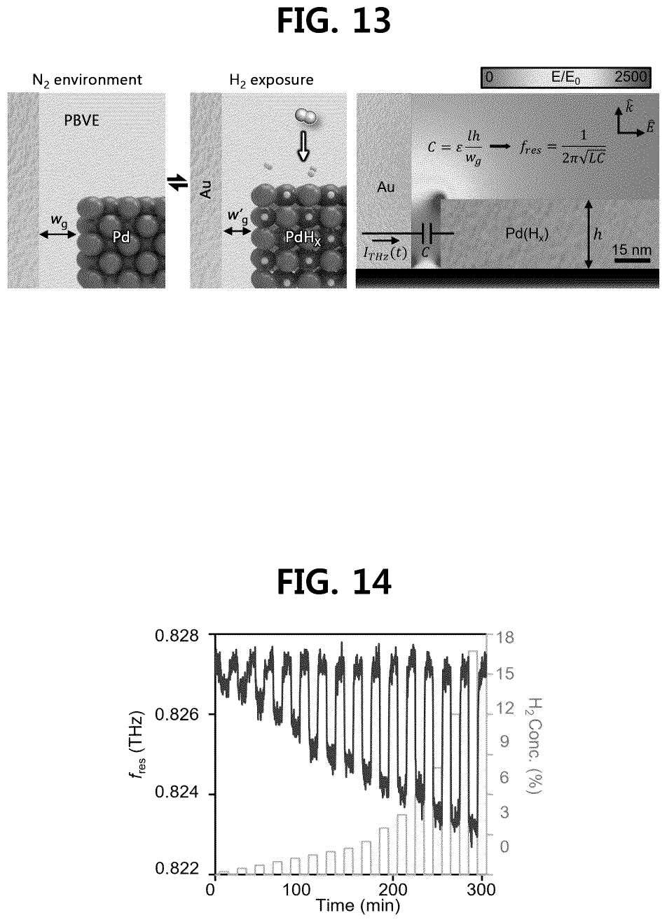

As shown in FIGS. 11 to 13, a very strong field enhancement is formed by the nanogap 155 formed between the wall surface of the metal 120 forming the metal nanoslot 130 and the catalyst layer 150.

The very strong field reinforcement formed by the nanogap 155 responds very sensitively to the thickness of the nanogap 155 and the catalyst layer 150.

As such, the field reinforcement formed by the nanogap 155 is affected by a capacitance effect between the catalyst layer 150 and the wall surface of the metal 120 forming the metal nanoslot 130, wherein the capacitance effect is affected by the conductivity of the wall surface of the metal 120 and the catalyst layer 150 and the distance between them.

Accordingly, it is possible to detect hydrogen by measuring the change in electrical properties due to the phase change of the catalyst layer 150 and the change in the distance between the wall surface of the metal 120 and the catalyst layer 150 due to volume expansion.

In an embodiment of the present invention, hydrogen can be detected by measuring the change in distance between the catalyst layer 150 and the wall surface of the metal 120, wherein the change in distance between the catalyst layer 150 and the wall surface of the metal 120 can be measured by measuring the transmittance and resonance frequency shift of the terahertz beam irradiated to the nanogap-based hydrogen sensor 100.

That is, when the beam receiving unit 230 receives the terahertz beam transmitted through the hydrogen sensor 100 via the first optical path (A), a signal processing unit 240 may measure the terahertz beam received through the first optical path (A) and obtain it as a signal for time.

FIG. 14 is a diagram exemplarily illustrating a change in a signal of a terahertz beam received through a first optical path (A) according to hydrogen concentration.

The signal processing unit 240, which has measured the terahertz beam received through the first optical path (A) and obtained it as a signal for time, may convert the signal for time into a signal for frequency through Fourier transform.

Here, a resonant signal of the terahertz beam transmitted through the hydrogen sensor 100 via the first optical path (A) is measured by a resonant phenomenon.

Thereafter, a signal analysis unit 250 may analyze the resonance frequency signal applied from the signal processing unit 240 to detect whether the catalyst layer 150 has been exposed to hydrogen.

Specifically, the signal analysis unit 250 may calculate the value by interpreting the maximum transmission value of the resonance frequency signal obtained from the terahertz beam transmitted through the hydrogen sensor 100 and the spectral value (frequency) at that value as variables. As the hydrogen concentration increases, the resonance frequency and maximum transmission value basically show a red shift and a decrease as shown in FIG. 15.

That is, when the hydrogen gas concentration in the gas chamber 200 increases as the hydrogen gas is injected into the gas chamber 200, it can be confirmed that the resonance frequency and maximum transmission value analyzed by the signal analysis unit 250 decrease in proportion to the hydrogen gas concentration.

As such, as the resonance frequency and the maximum transmission value are reduced in proportion to the hydrogen gas concentration, it is possible to reversely estimate the hydrogen concentration in the gas chamber 200 using the reduced resonance frequency.

Meanwhile, the nanogap-based hydrogen sensor 100 according to the present invention may function as an oxygen sensor by adsorption of oxygen as well as hydrogen.

This may be based on a mechanism in which the electrical conductivity increases due to oxygen adsorption on the surface of the catalyst layer 150 as the oxygen concentration increases.

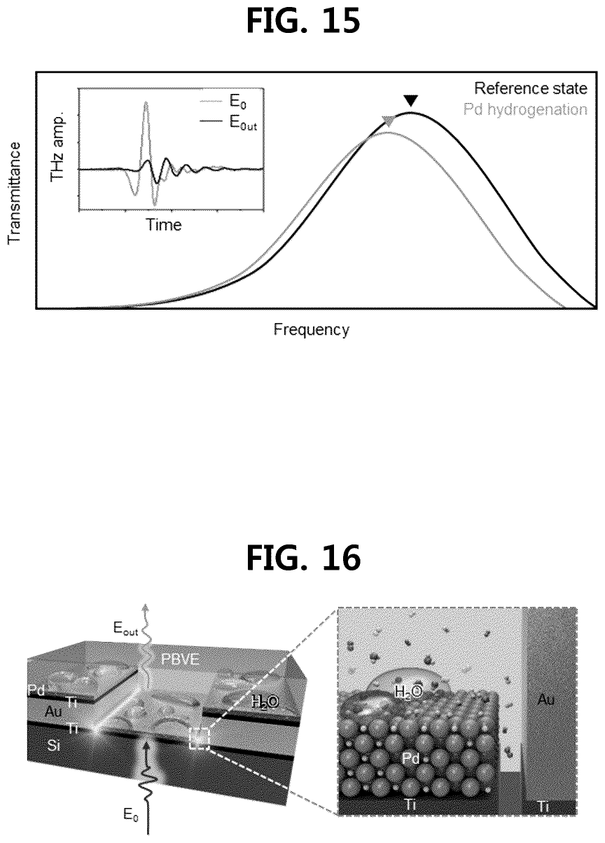

Specifically, when hydrogen gas (H₂) in the air is adsorbed on the surface of the catalyst layer 150 and the hydrogen gas adsorbed on the surface of the catalyst layer 150 combines with oxygen in the air, water may be generated on the surface of the catalyst layer 150 as shown in FIG. 16, and oxygen can be detected by observing the water generated on the surface of the catalyst layer 150.

In this case, as a strong field enhancement is formed by the nanogap 155 formed between the wall surface of the metal 120 forming the metal nanoslot 130 and the catalyst layer 150, it is possible to observe the generation of water at a nano level.

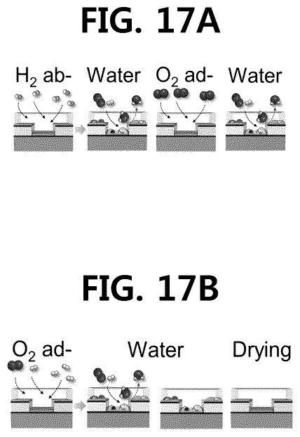

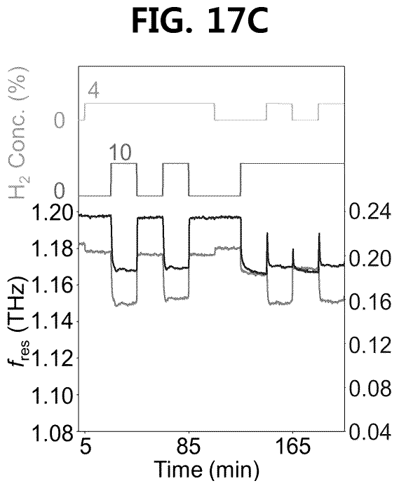

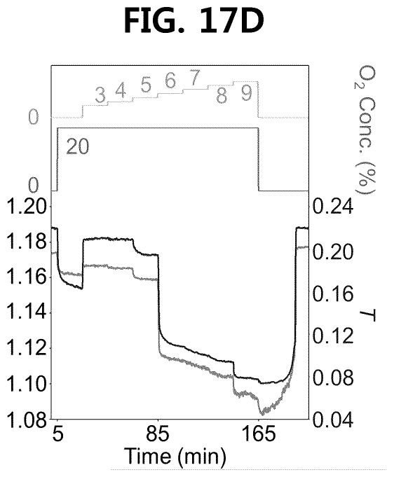

As described above, as the hydrogen gas adsorbed on the surface of the catalyst layer 150 is combined with oxygen in the air to generate water on the surface of the catalyst layer 150, the dynamics for the water generation can be tracked according to the time resolution, as shown in FIGS. 17A to 17D.

FIGS. 17A and B are diagrams schematically illustrating the dynamics between the catalyst layer 150 and the gas occurring in each gas atmosphere, and FIGS. 17C and 17D are diagrams illustrating data in which a reaction occurring according to a specific concentration of hydrogen and oxygen is measured as a signal, wherein the dynamics occurring under various concentrations can be observed by measuring the reaction between the resonance frequency and transmittance.

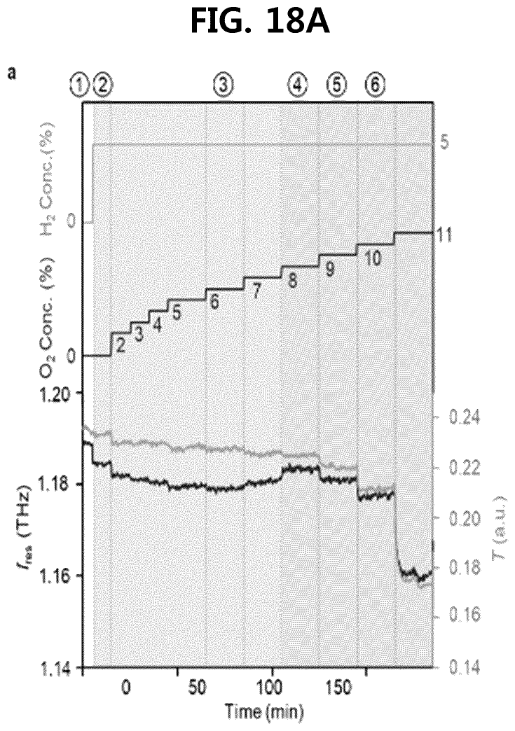

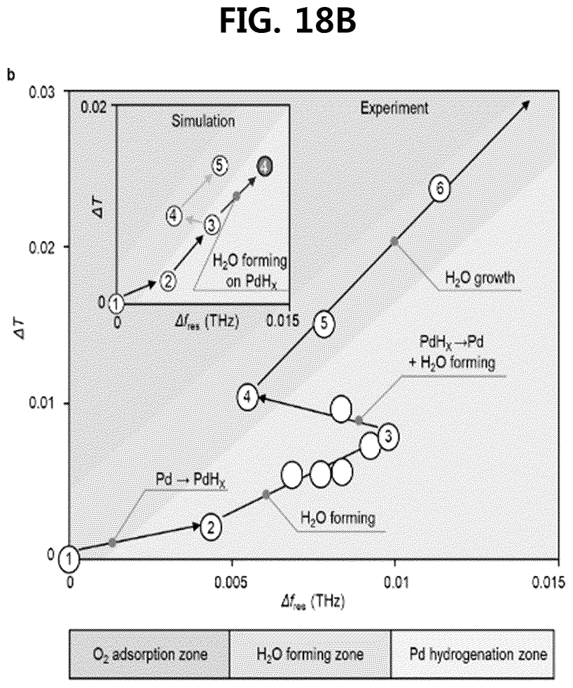

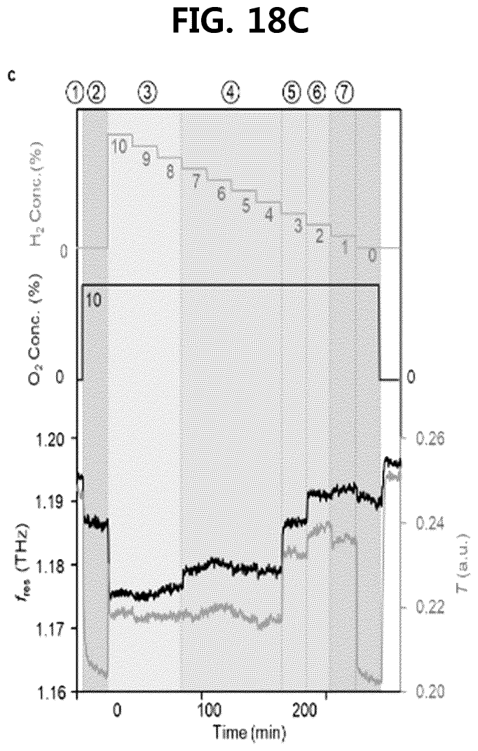

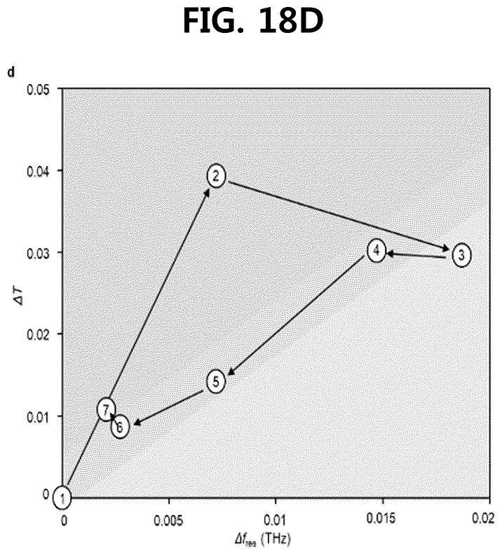

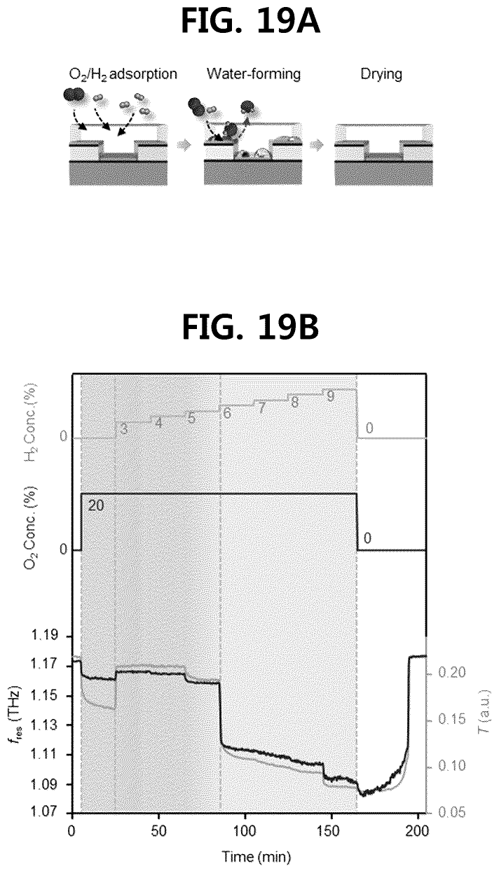

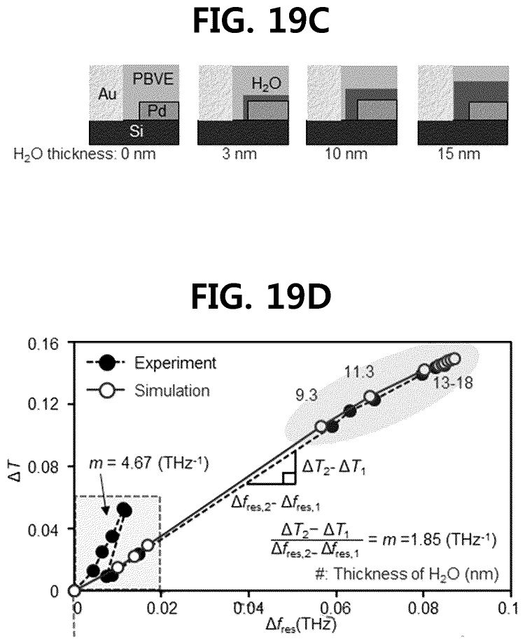

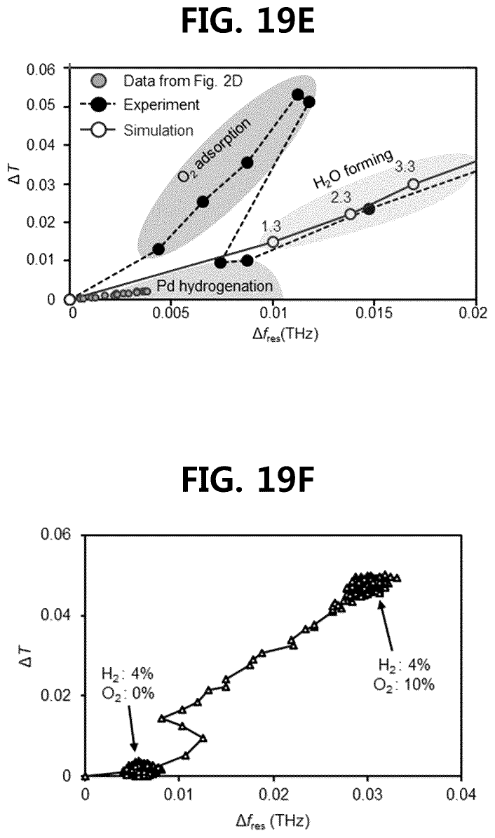

The dynamics for water generation can be tracked using terahertz time-based spectroscopy technology, as in the case where the nanogap-based hydrogen sensor 100 functions as a hydrogen sensor, and various dynamics can be interpreted using the frequency change and transmittance change according to the oxygen concentration change as variables, as shown in FIGS. 18A to 18D and FIGS. 19A to 19F.

FIGS. 18A to 18D show datas in which the dynamics for water generation are tracked in two main different environments, in which data (a) represents data measured in a state in which the oxygen concentration is gradually increased while the hydrogen is fixed at a certain concentration, and data (b) represents data in which the response is plotted in a region with changes in frequency and transmittance as axes. Through the data (b), it is possible to visualize the generation ((\textcircled{2})-\textcircled{3}) and growth (\textcircled{4}-\textcircled{6}) of water and the change process (\textcircled{3}-\textcircled{4}) of the catalyst layer 150 during the water generation process.

Similarly, data (c) represents data measured in a situation where the oxygen concentration is constant, and data (d) represents data in which the response is plotted in a region with changes in frequency and transmittance as axes. Through the data (d), it is possible to visualize the adsorption (\textcircled{1}-\textcircled{2}) and desorption (\textcircled{2}-\textcircled{3}) of oxygen, the process of water generation and reduction (\textcircled{4}-\textcircled{6}), and the change process of the catalyst layer 150 (\textcircled{3}-\textcircled{4}).

FIG. 19A is a schematic diagram showing the adsorption of hydrogen and oxygen, water generation, and drying process, and FIG. 19B shows data on a signal response according to a gradual increase in hydrogen concentration under an oxygen concentration of 20%. FIG. 19D shows data in which data for each time section in FIG. 19B is plotted in a region with changes in frequency and transmittance as axes. In FIG. 19D, the black points are data for each section in FIG. 19B, and the red points represent data obtained through the simulation results as shown in FIG. 19C. It can be confirmed that measurements can be made according to the thickness of the water by matching the simulated red data and the black experimental data according to the thickness of the water.

FIG. 19E is an enlarged data of a small region in FIG. 19D, in which a signal by oxygen adsorption (red region), a signal by water generation (blue region), and a signal by hydrogenation of the catalyst layer 150 (black region) can be observed separately.

FIG. 19F shows data in which data with a time resolution of approximately 3 seconds when oxygen is suddenly injected from 0% to 10% under a hydrogen concentration of 4% is plotted in a region with changes in frequency and transmittance as axes, and shows that the processes described above can be dynamically observed even under sudden changes in concentration.

As described above, the nanogap-based hydrogen sensor 100 according to an embodiment of the present invention can detect hydrogen by measuring the change in electrical properties due to the phase change of the catalyst layer 150 according to reaction with hydrogen, and the change in the distance between the wall surface of the metal 120 and the catalyst layer 150 due to volume expansion. Further, it can detect oxygen by observing that oxygen is adsorbed on the surface of the hydrogen sensor 100 to generate water, and can also detect gases other than hydrogen and oxygen.

As described above, according to the present invention, it is possible to detect a trace amount of hydrogen gas leakage through the nanogap formed between the catalyst layer and the metal wall surface.

In addition, since a beam in the terahertz band is irradiated to a hydrogen sensor with a nanogap formed and a signal transmitted through the hydrogen sensor is measured to detect hydrogen leakage, electrode patterning is not required and a high detection speed of less than tens of seconds is achieved.

In addition, since a micrometer-scale metal nanoslot pattern is formed on the wafer through a photolithography process, and a catalyst material is deposited on the surface of the metamaterial 140 through a thermal evaporator, it is possible to minimize differences between devices and mass-produce devices with uniform performance.

Additionally, it is possible to inversely estimate the concentration of hydrogen by measuring the shift of resonance frequency with low noise.

In addition, one nanogap-based hydrogen sensor can be used to detect not only hydrogen but also oxygen.

Although various preferred embodiments of the present invention have been described above by way of some examples, the descriptions of various embodiments described in the “DETAILED DESCRIPTION OF THE INVENTION” are merely illustrative, and it will be understood from the above descriptions by those of ordinary skill in the art to which the present invention pertains that the present invention may be implemented with various modifications or may be implemented equivalently to the present invention.

It should also be noted that the present invention is not limited by the above description because it can be implemented in various other forms; the above description is only provided to make the disclosure of the present invention complete and to fully inform those skilled in the art of the scope of the invention; and the present invention is only defined by the scope of each of the claims.

### DESCRIPTION OF REFERENCE NUMERALS

100: nanogap-based hydrogen sensor
110: wafer
120: metal
130: metal nanoslot
140: metamaterial
150: catalyst layer
155: nanogap
160: protective layer
200: gas chamber
210: light source unit
220: beam splitter
230: Beam receiving unit
240: signal processing unit
250: signal analysis unit
300: MFC

What is claimed is:

1. A nanogap-based hydrogen sensor including:
a metamaterial manufactured by forming a metal nanoslot pattern on a wafer;
a catalyst layer deposited on the surface of the metamaterial to form a nanogap inside the metal nanoslot; and
a protective layer formed on the catalyst layer to protect the catalyst layer.

2. The nanogap-based hydrogen sensor according to claim 1, wherein when the catalyst layer is formed by depositing a catalyst material on the surface of the metamaterial, the nanogap is formed between the metal wall surface and the catalyst layer inside the metal nanoslot due to the step difference of the metal nanoslot.

3. The nanogap-based hydrogen sensor according to claim 2, wherein the nanogap has a width of 10 to 25 nm.

4. The nanogap-based hydrogen sensor according to claim 1, wherein the catalyst layer causes a distance change from the wall surface of the metal forming the metal nanoslot as the volume thereof expands due to a reaction with hydrogen, thereby changing a gap size of the nanogap.

5. The nanogap-based hydrogen sensor according to claim 1, wherein the metal nanoslot pattern formed on the wafer is formed through a photolithography process.

6. The nanogap-based hydrogen sensor according to claim 1, wherein the catalyst layer is formed by depositing a catalyst material on the surface of the metamaterial through a thermal evaporator.

7. A method of manufacturing a nanogap-based hydrogen sensor, the method including the steps of:
manufacturing a metamaterial by forming a metal nanoslot pattern on a wafer;
depositing a catalyst material on the surface of the metamaterial to form a catalyst layer forming a nanogap inside the metal nanoslot; and
forming a protective layer protecting the catalyst layer on the catalyst layer.

8. The method of manufacturing a nanogap-based hydrogen sensor according to claim 7, wherein in the step of forming the catalyst layer, when the catalyst layer is formed by depositing a catalyst material on the surface of the metamaterial, the nanogap is formed between the metal wall surface and the catalyst layer inside the metal nanoslot due to the step difference of the metal nanoslot.

9. The method of manufacturing a nanogap-based hydrogen sensor according to claim 7, wherein the step of manufacturing the metamaterial is a step of manufacturing the metamaterial by forming the metal nanoslot pattern on the wafer through a photolithography process.

10. The method of manufacturing a nanogap-based hydrogen sensor according to claim 7, wherein the step of forming the catalyst layer is a step of forming a catalyst layer forming a nanogap inside the metal nanoslot by depositing a catalyst material on the surface of the metamaterial through a thermal evaporator.
